(19) United States
(12) Patent Application Publication
(10) Pub. No.: US 2010/0030076 A1
(43) Pub. Date: Feb. 4, 2010

(54) Systems and Methods for Simultaneously Treating Multiple Target Sites

(76) Inventors: Kobi Vortman, Haifa (IL); Shuki Vitek, Haifa (IL); Oleg Prus, Haifa (IL)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/509,907

(22) Filed: Jul. 27, 2009

Related U.S. Application Data
(63) Continuation-in-part of application No. 11/461,763, filed on Aug. 1, 2006.

(51) Int. Cl. A61B 8/00; A61N 7/00

(52) U.S. Cl. 600/439; 601/2

(57) ABSTRACT

The emission intensities of groupings of transducer elements of an ultrasound transducer array are controlled based on targeting criteria in such a manner as to simultaneously create multiple discontiguous foci, each corresponding to one of a plurality of target sites.

### CROSS-REFERENCE TO RELATED APPLICATIONS

This application is a continuation-in-part of co-pending U.S. patent application Ser. No. 11/461,763, entitled “Ultrasound Transducer with Non-Uniform Elements” filed on Aug. 1, 2006, the entire disclosure of which is hereby incorporated by reference herein.

### FIELD OF THE INVENTION

The field of the invention relates generally to thermal and mechanical energy treatment systems and, more particularly, to systems and methods for controlling the intensity of acoustic energy transmitted from an array of transducer elements in a manner as to simultaneously produce multiple foci, each directed at a different target site.

### BACKGROUND

High-intensity focused acoustic waves, such as ultrasound or acoustic waves at a frequency greater than about 20 kilohertz, may be used to therapeutically treat tissue regions within a patient. For example, ultrasound waves may be used in applications involving ablation of tumors, thereby eliminating the need for invasive surgery, targeted drug delivery, control of the blood-brain barrier, lysing of clots, and other surgical procedures.

Focused ultrasound systems typically include piezoelectric transducer elements (also referred to herein as “elements”) that are driven by electric signals to produce ultrasound energy. In such systems, a transducer may be geometrically shaped and positioned such that ultrasound energy emitted by an array of transducers collectively forms a focused beam at a “focal zone” corresponding to the target tissue region. As used herein, the terms “beam,” “energy beam,” or “acoustic energy beam” refer generally to the sum of the waves emitted by the various transmitting elements of a focused ultrasound system.

High-intensity focused ultrasound treatments direct the acoustic beam at the target area to achieve intensities or power densities that are high enough to destroy tissue, e.g., via coagulation or non-thermal mechanical effects. However, tissue along the acoustic beam path also absorbs energy (albeit at significantly lower intensities), so that each sonication induces a slow temperature rise in the non-targeted tissue. In conventional methods, the transmission of the acoustic beam is halted periodically to allow the tissue in the path zone to cool down to a baseline temperature. Since the cooling is achieved by perfusion and diffusion, which are slow processes, the need for cooling periods significantly increases the overall treatment time, which in turn limits the adoption of focused ultrasound as a preferred method of treatment. In most instances the heating/treatment rate of targeted tissue is limited by the need to minimize heating of the non-targeted tissue. Therefore, if heating of non-targeted tissue could be significantly reduced or even eliminated, acoustic energy could be delivered more or less continuously, thus decreasing the treatment time.

Tissue heating rates depend on the intensity (energy density) of the acoustic beam applied to the tissue. This implies that reducing the intensity in the beam path zone will reduce treatment times. Further, because the intensity is inversely proportional to the transducer area, using a transducer having a large area could reduce the energy density in the path zone (and, hence, treatment times). But because transducer elements have a finite size and the beams generated by the elements have particular directionalities, the energy contribution of a particular element diminishes as the steering angle relative to the target zone increases; in particular, elements having a high steering angle with respect to the target provide a limited contribution to the intensity at the focus site, thus introducing a significant amount of “ineffective” energy into the volume. In these situations where only part of the transducer area is effectively contributing to energy reaching the focus, the non-contributing elements are typically switched off—i.e., their potential contributions are effectively wasted.

It would be beneficial, therefore, to utilize the transducer elements not being directed at a lesion or target area to simultaneously deliver focused ultrasound to additional target areas.

### SUMMARY

Embodiments of the invention provide techniques and systems that facilitate the simultaneous application of focused ultrasound to multiple target sites in a manner that reduces overall treatment time while avoiding harm to healthy anatomy outside the target zones. More specifically, a transducer surface is segmented into sub-areas (also referred to as “element groupings”), each of which results in a separate focus directed to a different target area. To maintain independence among the groupings, a maximum allowable (or in some cases no) beam path zone overlap is adhered to. The ability to simultaneously treat multiple foci (e.g., multiple nodules or tumors) greatly accelerates the treatment rate, and therefore overall acceptance of focused ultrasound as a treatment modality.

Thus, in a first aspect of the invention, a system for delivering acoustic energy to multiple target sites within a patient includes a transducer array comprising multiple transducers, each of which transmits acoustic energy to a respective focus. The foci are discontiguous (i.e., spatially distinct) and address a pattern of target sites. The system also includes a processor coupled to the array for establishing targeting criteria corresponding to the pattern and a controller coupled to the processor and the transducer elements for driving (e.g., providing excitation signals to) the transducer elements based on the targeting criteria.

In some embodiments, the transducer array includes a plurality of grouped transducer elements based on the targeting criteria, and each group may produce ultrasound energy at different frequencies and/or have different focal lengths. The groups may be activated (and thus deliver acoustic energy) simultaneously, or, in some cases, quasi-simultaneously in rapid-switching fashion. The transducer groups, in other words, can be selected and grouped ad hoc from a set of available transducer elements based on the desired targeting geometry. That geometry may include steering angles of the transducer elements with respect to each of the target sites, lines of sight from the transducer elements to each of the target sites, anatomical features of the patient, locations of the target sites within the patient, and/or f-numbers (i.e., the focal length divided by an emitting area) of the transducer elements.

Each group of transducer elements may be independently controllable, and may include a single beamformer for driving each of the transducer element groups, or a multiple beamformers, each driving one of the transducer element groups. In some cases, the transducer element groups are mechanically connected and/or flexibly connected to allow for contortion of the array about a patient. In some implementations, the element groupings may be defined such that the lines of sight from each element group to its corresponding target site do not pass through a previously-identified no-pass region and/or such that any overlap of the lines of sight from each element group to its corresponding target site remains below an overlap threshold.

The system may, in some embodiments, also include an imager for capturing a single image that includes all the target sites, a series of images of the multiple target sites, or in some implementations, multiple imagers are used to simultaneously generate multiple images of the target sites.

In another aspect of the invention, a method for simultaneously delivering focused ultrasound to multiple target sites includes the steps of providing an ultrasound transducer array comprising multiple transducer elements and determining targeting criteria for each element with respect to the target sites. The method also includes determining element groupings based on the targeting criteria and driving the transducer elements based upon the element groupings in order to simultaneously focus acoustic energy transmitted by the transducer elements at the different target sites.

In some embodiments, multiple independent transducer arrays may be mechanically connected to form the ultrasound transducer array. The targeting criteria may include, for example, steering angles of the transducer elements with respect to each of the target sites, lines of sight from the transducer elements to each of the target sites, and/or f-numbers of the transducer elements. The groups may be activated (and thus deliver acoustic energy) simultaneously, or, in some cases, quasi-simultaneously in rapid-switching fashion. In some cases, a no-pass region (or regions) may be defined through which no ultrasound energy is permitted to pass and the element groupings are determined such that the lines of sight from each element grouping to its corresponding target site do not pass through the no-pass region(s). In still other cases, the element groupings may be defined such that any overlap of the lines of sight from each element grouping to its corresponding target site remains below an overlap threshold. Images of the various target zones may be taken to confirm the delivery of ultrasound energy to the target zones.

In another aspect, the invention relates to an article of manufacture having computer-readable program portions embodied therein for authentication using biometrics. The article comprises computer-readable program portions for performing the method steps described above.

### BRIEF DESCRIPTION OF THE DRAWINGS

In the drawings, like reference numbers represent corresponding parts throughout.

FIG. 1 schematically illustrates a patient with multiple lesions to be treated using focused ultrasound.

FIG. 2 is a schematic section illustrating the application of an ultrasound transducer array to facilitate the application of ultrasound energy to the patient.

FIG. 3 schematically illustrates an array of focused ultrasound transducer elements grouped based on one or more targeting criteria according to one embodiment of the invention.

FIG. 4 schematically illustrates the determination of the steering angle of a transducer element.

FIG. 5 is a schematic section illustrating the application of the array of FIG. 3 to a patient according to one embodiment of the invention.

FIG. 6 is a schematic of a system for delivering focused ultrasound energy to multiple lesions within a patient according to various embodiments of the invention.

FIG. 7 schematically illustrates a plurality of mechanically-connected focused ultrasound transducer arrays according to one embodiment of the invention.

FIG. 8 is a flow chart of a technique for simultaneously applying focused ultrasound to multiple lesions within a patient according to various embodiments of the invention.

### DETAILED DESCRIPTION

High-density ultrasound transducers may utilize a two-dimensional grid of uniformly shaped piezoelectric (PZT) “rods” glued to a conductive matching layer substrate. For both manufacturing and performance reasons, the PZT rods typically have rectangular (or square) profiles, with an aspect ratio (i.e., ratio of height to width) of greater than or equal to one, and are preferably uniform in size to produce the same frequency response. Spacing between the rods also influences the acoustic performance of the transducer and is preferably minimized such that it is smaller than the size of the rods themselves. A high-density phased-array transducer may have hundreds, even thousands of densely packed piezoelectric rods, each having a relatively small (e.g., 1 mm²) energy transmitting surface.

Such transducer arrays have been used to apply acoustic energy to patients for both imaging and therapeutic purposes. Typical therapeutic applications provide a focused beam of acoustic energy to a single focal point (usually within the boundaries of a lesion or tumor) in order to ablate the diseased tissue. The ability to focus the ultrasound at small, well-defined regions by varying the driving signals of the transducer elements permits the targeting of small lesions embedded deep within a patient while avoiding excessive heating of (and consequent damage to) healthy tissue. However, if multiple lesions are to be treated, the transducer is ordinarily applied to the patient separately for each lesion, requiring time-consuming parameter and equipment adjustments.

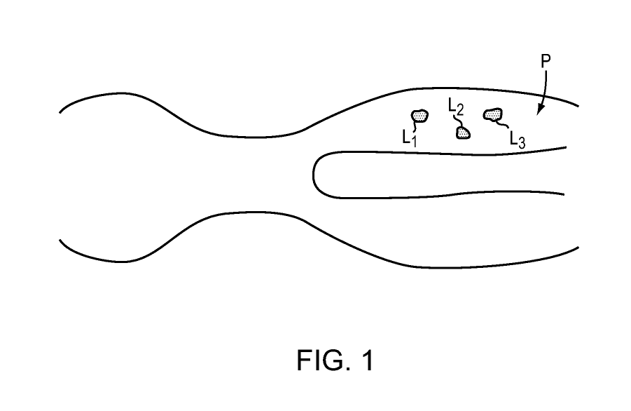

Referring now to FIG. 1, a patient P has multiple lesions L₁, L₂ and L₃ (referred to generally as L) that are to be treated using focused ultrasound. Although the lesions L are illustrated as being embedded within the patient P, one or more of the lesions L may be on the body's surface (e.g., skin cancer lesions, moles, or other topical growth).

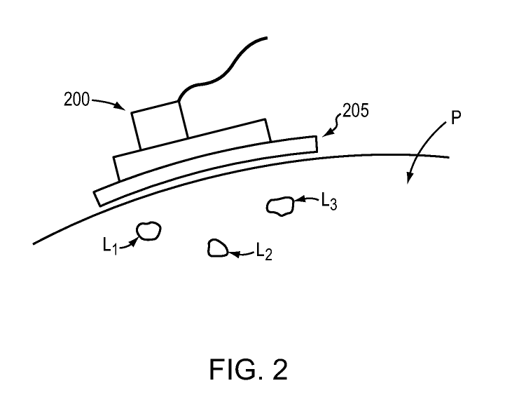

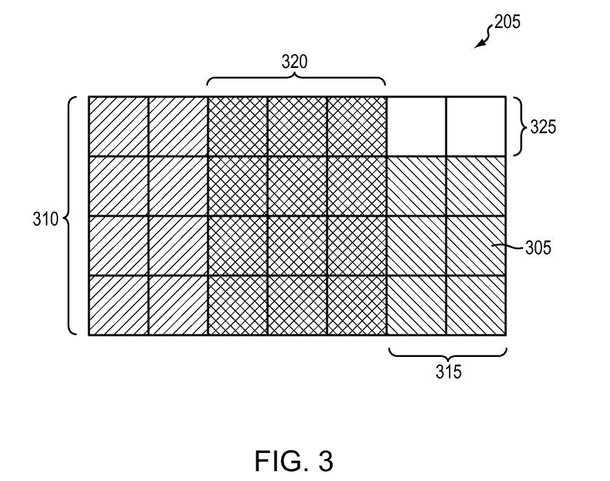

FIG. 2 illustrates the application of an ultrasound delivery device 200 to the patient P. The device 200 includes an array of transducer elements 205 that, when connected to and driven by a controller (not shown), deliver acoustic energy to the patient P. According to various embodiments of the invention, the high-density transducer array 205 includes piezoelectric rods (also referred to herein as “elements”) as shown in FIG. 3. The transducer array 205 comprises a two-dimensional arrangement of individual elements 305 affixed (e.g., glued) to a planar substrate. The elements 305 may be substantially identical in size and shape, including having substantially uniform (square) distal facing energy-transmitting surfaces. In some embodiments, the elements 305 are non-uniform. The elements 305 may be arranged in uniformly aligned columns and rows, with minimal spacing provided between adjacent rods. It will be appreciated that the relatively small transducer size allows for greater electronic steering capability of the overall array.

In some embodiments, each element may be connected to its own electronic drive signal input, such that each element forms a distinct transducer element that can operate independently of the others, and the elements 305 may therefore be grouped arbitrarily. The acoustic attributes (e.g., frequency response, efficiency, etc.) of the array 205 are influenced by the three-dimensional structure of the individual elements 305, and preferably the height of each element is equal or greater than its width. However, the steering/focusing ability of the transducer array 205 is fully defined by the geometric surface (i.e., the overall area of the transducer elements that emit respective acoustic waves with the same phase) of the respective elements 305.

Still referring to FIG. 3, and according to various embodiments of the invention, multiple elements 305 may be grouped into one or more groupings 310, 315 and 320 in order to simultaneously target individual, spatially separated lesions. In some cases, some elements may be disabled (as indicated at 325), while the other groupings 310, 315 and 320 deliver acoustic energy according to various targeting criteria. In certain embodiments, the surface shapes of the transducer elements may have rectilinear or curvilinear profiles, or a combination of both, and/or may include many different types of “irregular” shapes (e.g., an L-shape or a T-shape).

The groupings may be determined by one or more targeting criteria that specify the geometric relationships among the elements 305 and/or between the elements 305 and the target sites (e.g., steering angles and/or lines of sight). The targeting criteria may also consider the physical locations of the target areas, the number of target areas, anatomical features within the target areas (or surrounding areas, such as vital organs) as well as characteristics of the elements themselves.

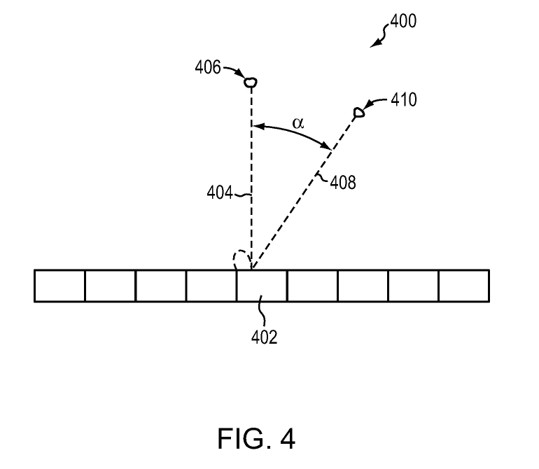

As an example, FIG. 4 illustrates the principle of electronic steering of a two-dimensional planar transducer array 400 that includes numerous uniformly shaped and arranged elements (such as elements 305 of FIG. 3). In particular, the “steering angle” of any one transducer element 402 of the array 400 is the angle α between a first focal axis 404 extending generally orthogonally from the element to an “unsteered” focal zone 406 at which the element 402 contributes a maximum possible power, and a second focal axis 408 extending from the transducer element 402 to a “steered-to” focal zone 410. The “steering ability” of the transducer array 205 is defined as a steering angle α at which energy delivered to the steered-to focal zone 410 from a given one-dimensional element row falls to half of the maximum power delivered to the unsteered focal zone 406. Notably, the steering angle of each transducer element of a phased array may be different, but as the distance from the elements to the focal zone increases, the respective steering angles for the array elements approach the same value.

From a physical point of view, a single transducer element emits a wave in the form of a spreading beam. The angular distribution of this spreading beam is called “directivity.” While a single small element of an array (if it is the only element that is activated) may not produce a focused beam, an array of activated elements can produce a focused beam, where the size of the “focus” is smaller when the combined transducer elements form a larger emitting surface. Each transducer element contributes to the focus proportionally based on its directivity at the “focus” and the power it transmits. Thus, the steering region of a phased-array transducer depends on each element's directivity patterns.

The relationship between an element's surface size and its steering ability can be represented in terms of its half-energy angle. For example, a transducer element may have a size-to wavelength value of /d/λ, where d is the size of the element in one dimension (e.g., width) and λ is the wavelength of the wave emitted by the element. In such a case, the half energy steering angle, or “steering ability,” of the transducer array with dλ=1 is 30°. This represents the angle at which a steered-to focal zone has an energy level equal to half the maximum energy that the transducer would contribute to a unsteered focal zone.

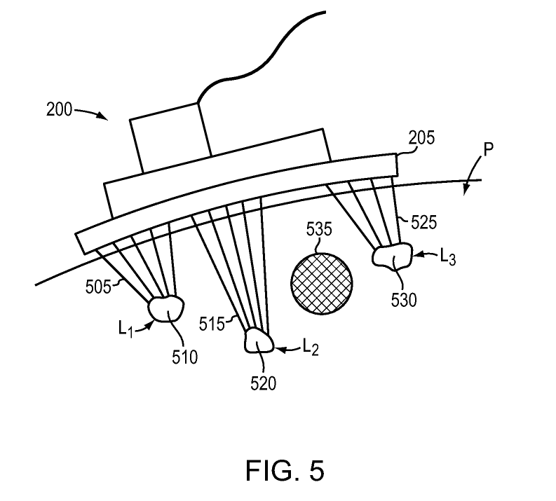

Referring to FIG. 5, a high-density, two-dimensional transducer array 205 having multiple element groupings is used to simultaneously direct acoustic energy to multiple discontiguous focal points. For example, a first element grouping creates a beam 505 directed at a first focal point 510 within (or in some cases near) a first lesion L₁. Likewise, a second element grouping creates second focused beam 515 directed at a second focal point 520 within a second lesion L₂. A third element grouping creates third focused beam 525 directed at a third focal point 530 within a second lesion L₃. In some cases, a “no-pass-zone” 535 may be identified (representing, for example, a vital organ or healthy tissue) and the element groupings determined such that none of the beams passes through the zone. In some cases, the array may be constructed as having a curved surface area, thus created a three-dimensional array.

By “discontiguous” is meant that the focal points are spatially distinct. In some instances the foci are sufficiently separated in space that the beam paths and the affected tissue regions around the foci are also spatially distinct, i.e., do not overlap. In other instances, however, the locations of the lesions L may be such that the beams from two or more element groupings overlap (i.e., more than one beam passes through certain tissue). A small amount of beam overlap may be acceptable, but larger amounts may cause unwanted accumulated heating of non-target tissue or interference. Therefore, in some embodiments, overlap thresholds are established and used to limit the amount (in terms of energy density, time or both) that two or more beams may pass through the same tissue. One example of an energy density threshold for a particular organ or anatomical region is 500 joule/cm².

In some embodiments, the multi-foci targeting may be implemented in quasi-simultaneous fashion using, for example, rapid switching. In such cases, the element groupings may be activated and deactivated according to a timed sequence so that the acoustic energy is delivered to each of the multiple foci in turn, albeit during a single application. The grouping of transducer elements into different sectors of the entire array may be exclusive, or, in some cases overlap such that some transducer elements are assigned to more than one group. In certain cases, some transducer elements may be ignored completely, if, for example, the trajectory of the acoustic energy is beyond the critical angle at which the contribution of the element is negligible. The selection and execution of a timing pattern may be based, for example, on an analysis of the acoustic path leading from the transducer array to the focal points, the size and/or shape of the desired foci, the depth of the desired foci, as well as other treatment parameters.

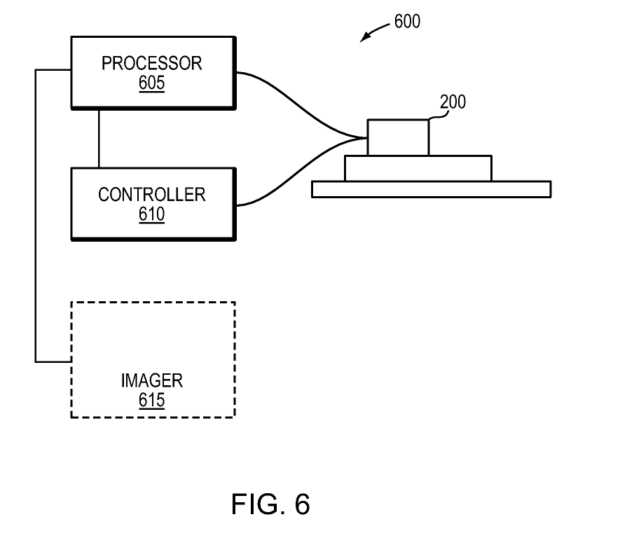

Referring to FIG. 6, a representative system 600 for determining the element groupings and driving the transducer elements includes the transducer array 205, a processor 605 for determining the targeting criteria based on the number and arrangement of the targets to be treated, and a controller 610 for driving the transducer elements according to the targeting criteria.

The array 205 may be constructed, by way of example and not limitation, using a conventional dicing machine, but making much smaller cuts to create a uniform array of piezoelectric elements in the same formation as shown in FIG. 3. The individual elements may be coupled to a same electronic drive signal in order to form the element groupings of the array 205. In other embodiments, the array may be formed by combining multiple (as an example four) previously independent arrays (referred to herein as “sub-arrays”) into a single device. In some cases, each of the previously independent arrays may receive its own drive signal. In such cases, each of the arrays may be connected to a common processor and controller to permit coordination of signals across the multiple arrays. For example, if four arrays are combined into a single unit, the controller is configured to provide four separate sets of drive signals—one set for each “sub-array” such that the transducer elements within each sub-array target the appropriate site. A single beamformer may be used for all the groups, or, in some cases, separate beamformers may be used for each of the groupings. Drive circuitry is coupled to the transducer elements, and provides respective drive signals to the transducer elements, whereby the transducer elements emit acoustic energy from their respective acoustic emission surfaces. In some cases (e.g., instances where the number of sub-arrays is greater than the number of lesions or target regions) the processor 605 may determine that multiple sub-arrays may be grouped together and aimed at a single lesion.

The electronic controller 610 is coupled to the drive circuitry and controls phase-shift values and amplitudes of the respective drive signals to further focus the acoustic energy emitted by the grouped transducer elements toward the different target regions. For example, the electronic controller may be configured to control phase-shift values of the drive signals to the transducer elements of the different groupings to simultaneously control the focal distances of the different acoustic energy beams emitted by the transducer element groupings. These parameters are determined and optimized to fullfil a particular set of targeting criteria.

The element groupings and the phase-shift values are determined based on one or more targeting criteria by the processor 605. In particular, the processor may receive information related to the arrangement of the transducer elements within the array, the elements' geometry, elements frequency response, the number and locations of the target areas (with respect to the array, each other, other anatomical structures, or some combination thereof), and in some cases locations of “no-pass-zones” through which no acoustic energy is to be transmitted.

The processor 605 may be implemented in hardware, software or a combination of the two. For embodiments in which the functions are provided as one or more software programs, the program may be written in any one of a number of high level languages such as FORTRAN, PASCAL, JAVA, C, C++, C#, BASIC, various scripting languages, and/or HTML. Additionally, the software can be implemented in an assembly language directed to the microprocessor resident on a target computer; for example, the software may be implemented in Intel 80×86 assembly language if it is configured to run on an IBM PC or PC clone. The software may be embodied on an article of manufacture including, but not limited to, a floppy disk, a hard disk, an optical disk, a magnetic tape, a PROM, an EPROM, EEPROM, field-programmable gate array, or CD-ROM. Embodiments using hardware circuitry may be implemented on, for example, one or more FPGA, CPLD or ASIC processors for controlling the phase, frequency and amplitude of the respective elements.

In some embodiments, the system also includes an imager 615 for capturing and providing images of the lesions (and in some cases general anatomical information) to the processor. The images may be generated by multiple imagers in order to capture different lesions or target areas at the same time, or use a single imager to generate a series of images as the imager is scanning each target area or the whole anatomy. The imager may be, for example, a computed tomography (CT) image, a magnetic-resonance imager (MRI), an X-ray device or an ultrasound imager, or any other suitable medical imaging modality.

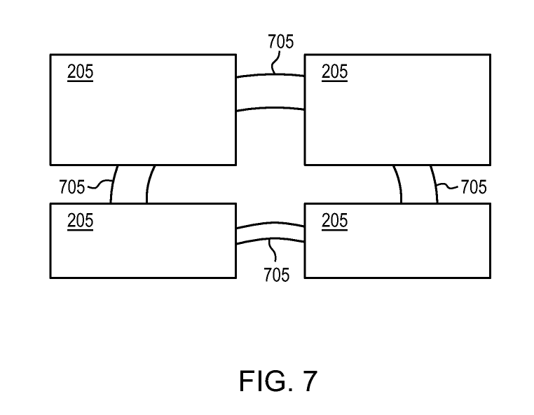

Referring to FIG. 7, multiple independent arrays 205 may be joined together to form a single array using various connectors 705. For example, some embodiments may utilize rigid connectors (e.g., rods or bars made of hard plastic or metal) to ensure the arrays 205 enforce a constant spacing and geometric arrangement. In other embodiments, the arrays may be joined using a flexible material (e.g., a fabric strap) to allow the arrays to move about each other. Such implementations permit the array to “fit” over a non-planar surface (e.g., a skull, abdomen or breast), in an orifice, or about a rounded appendage. Such maneuverability, when combined with the ability to simultaneously treat more than one lesion, permits a rapid administration of ultrasound energy to a patient without the need for adjustments or multiple positioning.

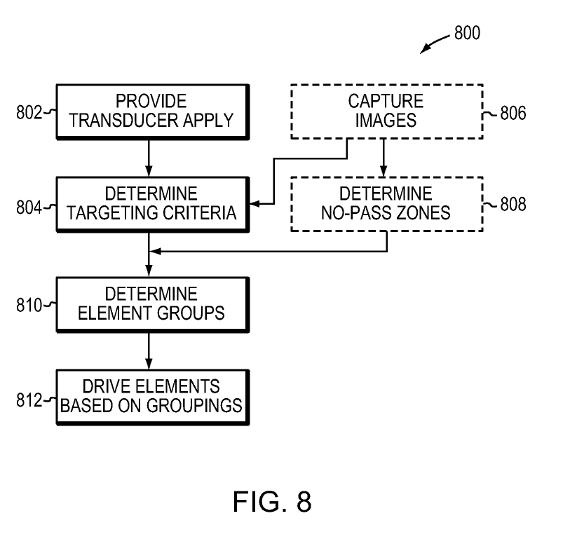

FIG. 8 illustrates a process 800 for simultaneously administering acoustic energy at multiple discontiguous target sites within a patient. Initially, a transducer array (or arrays) is provided (STEP 802), each including multiple transducer elements. Targeting criteria that relate the elements to each other (e.g., arrangement) and/or to the target areas are then determined (STEP 804). As described above, the targeting criteria may include the steering angles of the transducer elements with respect to each of the target sites, lines of sight from the transducer elements to each of the target sites, the number of target areas, overlap thresholds, and/or no pass zones. In some embodiments, one or more images may be taken (STEP 806) of the target sites to aid in determining the targeting criteria, locate the target areas and/or determine no-pass zones (STEP 808) through which no acoustic energy is to be transmitted. Based on the targeting criteria (and, in some embodiments, information received from the images), the elements are grouped (STEP 810) into two or more groups, where each group corresponds to one of the multiple target areas. Excitation signals are then sent to the transducer elements (STEP 812) such that the grouped drive elements operate together.

In some embodiments, before the transducer array is activated to deliver treatment-level ultrasound energy, an acoustic wave simulation is performed to determine if any hot spots will be generated. For example, a computer model of the transducer may be created to model the configuration (e.g., shape, size, and relative position) of the transducer elements. Various operational parameters (such as operation frequencies, amplitudes, and phases for the various transducer elements) can then be applied to the computer model to determine if a hot spot will result from a certain operational condition. As will be appreciated by those skilled in the art, while all transducer elements of an array may be activated in some instances, e.g., in order to maximize an amount of energy delivered to a steered-to target area, in other instances, sufficient therapeutic energy may be delivered without activating all elements of a group.

As used herein, the term “hot spot” refers to a tissue region that is not part of the target having an energy level (which may be measured, for example, in terms of temperature or acoustic pressure) that is above a prescribed (safe) level at which the tissue in the hot spot will be temporarily or permanently injured. Because such hot spot(s) start to appear as the electronic steering angle increases, electronic steering to each possible “steered-to” focal zone must be carefully analyzed for safety purposes before undertaken. Further, the energy absorbed at the hot spot(s) decreases the remaining energy available for contributing to the intended “steered-to” focal zone.

In order to better illustrate the relationship between the electronic steering angle and formation of hot spot(s), consider a one-dimensional array (i.e., row) of transducer elements having a cross sectional dimension scaled to wavelength (i.e., element surface size) of /d/λ=1. If Δφ is a phase difference between neighboring elements of the array, Δφ=d sin(α), maximum energy emission occurs at angles satisfying the relationship:

$$Δ ϕ = d \cdot sin ( α ) \cdot \frac{2 π}{λ} ,$$

where λ is an ultrasound wavelength, integer n=0 for the main focus and n≈0 for hot spots. Thus, where d≦λ/2, no hot spots will be formed. As such, the advantages of the embodiments described below particularly apply where the element size is equal to or greater than one-half of the drive signal wavelength.

The electronic steering ability of a transducer device can be defined as

$${I}_{s} ≡ \frac{Energy at main focus}{All emmited energy}$$

being above a preset threshold. For d>>λ, the steering ability approaches single-element directivity,

$${I}_{d} = {( \frac{sin ( π d sin ( α ) / λ )}{π d sin ( α ) / λ} )}^{2} .$$

As a result of hot-spot generation, large steering angles cannot be practically used where elements sizes are above 0.5λ, since nearly all of the energy that does not go to the steered-to focal zone is concentrated at hot spots. For d=λ, while attempting to steer to 30°, hot spots are produced at −30° of equal intensity as the main focus, reducing the steering ability that can be safely used to about half of the main focus steering ability. It will be appreciated by those skilled in the art that as the steering angle amplitude (absolute value) increases, hot spots begin to appear at numerous different points, and are both uncontrollable and undesirable.

Thus, although particular embodiments of the invention have been shown and described, it should be understood that the above discussion is not intended to limit the invention to these illustrated and described embodiments, which are provided for purposes of example only. Instead, the invention is defined and limited only in accordance with the following claims.

What is claimed is:

1. A system for delivering acoustic energy to a plurality of target sites within a patient, the system comprising:
a transducer array comprising a plurality of transducers each transmitting acoustic energy to a respective focus, the foci being discontiguous and forming a pattern of target sites;
a processor coupled to the array for establishing targeting criteria corresponding to the pattern; and
a controller coupled to the processor and the transducers, the controller driving the transducer elements based at least in part on the targeting criteria.

2. The system of claim 1 wherein each transducer comprises a plurality of grouped transducer elements, the element groupings being based on the targeting criteria.

3. The system of claim 1 wherein the targeting criteria comprise one or more of steering angles of the transducers with respect to each of the target sites, lines of sight from the transducers to each of the target sites, anatomical features of the patient, locations of the target sites within the patient or transducer elements shapes and dimensions.

4. The system of claim 3 wherein each group of transducer elements is controllable independently of the other groups.

5. The system of claim 4 wherein the transducer element groups are mechanically connected.

6. The system of claim 4 wherein the transducer element groups are flexibly connected.

7. The system of claim 4 further comprising a single beamformer for driving each of the transducer element groups.

8. The system of claim 4 further comprising a plurality of beamformers, each driving one of the transducer element groups.

9. The system of claim 3 wherein the element groupings are defined such that the lines of sight from each element group to its corresponding target site do not pass through a previously-identified no-pass region.

10. The system of claim 3 wherein the element groupings are defined such that any overlap of the lines of sight from each element group to its corresponding target site remains below an overlap threshold.

11. The system of claim 1 wherein the controller drives the transducer elements such that the acoustic energy is delivered to the foci in rapid switching fashion.

12. The system of claim 1 wherein each of the element groups produces ultrasound energy at different frequencies.

13. The system of claim 1 wherein each of the element groups has a preset focal length.

14. The system of claim 1 further comprising an imager for capturing images of the multiple target sites.

15. The system of claim 14 wherein the imager comprises a plurality of imaging devices, thereby simultaneously generating multiple images of the target sites.

16. The system of claim 14 wherein the imager comprises a single imaging device for generating a sequence of images capturing multiple images of the target sites.

17. The system of claim 14 wherein the imager comprises a single imaging device configured to generate a single image capturing multiple images of the target sites.

18. A method for simultaneously delivering focused ultrasound to a plurality of discontiguous target sites, the method comprising:
providing an ultrasound transducer array comprising a plurality of transducer elements;
determining one or more targeting criteria for each element with respect to the target sites;
determining element groupings based on the targeting criteria; and
driving the transducer elements based upon the respective element groupings to simultaneously focus acoustic energy transmitted by the transducer elements at the plurality of target sites.

19. The method of claim 18 further comprising mechanically connecting a plurality of independent transducer arrays to form the ultrasound transducer array.

20. The method of claim 18 wherein the transducer elements are driven in rapid switching fashion such.

21. The method of claim 18 wherein the targeting criteria comprise one or more of steering angles of the transducer elements with respect to each of the target sites, lines of sight from the transducer elements to each of the target sites, or f-numbers of the transducer elements groupings.

22. The method of claim 21 further comprising defining a no-pass region through which no ultrasound energy is permitted to pass and wherein the element groupings are determined such that the lines of sight from each element grouping to its corresponding target site do not pass through the no-pass region.

23. The method of claim 21 wherein the element groupings are defined such that any overlap of the lines of sight from each element grouping to its corresponding target site remains below an overlap threshold.

24. The method of claim 18 further comprising capturing one or more images of the target zones.
